SEQUENCE LISTING (5 sequences)

SEQ ID NO: 1 - DNA, 27 bp, Artificial Sequence
  Description of Artificial Sequence Synthetic polynucleotide sequence
gcgaggtaaaacgacggccagtctcgc

SEQ ID NO: 2 - DNA, 27 bp, Artificial Sequence
  Description of Artificial Sequence Synthetic polynucleotide sequence
tttttactggccgtcgttttactcttt

SEQ ID NO: 3 - DNA, 24 bp, Artificial Sequence
  Description of Artificial Sequence Synthetic polynucleotide sequence
cgtatcattggactggccatttat

SEQ ID NO: 4 - DNA, 17 bp, Artificial Sequence
  Description of Artificial Sequence Synthetic polynucleotide sequence
actggccgtcgttttac

SEQ ID NO: 5 - DNA, 17 bp, Artificial Sequence
  Description of Artificial Sequence Synthetic polynucleotide sequence
cgtatcattggactggc

(19) United States
(12) Patent Application Publication
(10) Pub. No.: US 2005/0112605 A1
(43) Pub. Date: May 26, 2005

(54) Reagentless, reusable, bioelectronic detectors and their use as authentication devices

(75) Inventors: Alan J. Heeger, Santa Barbara, CA (US); Kevin W. Plaxco, Santa Barbara, CA (US)

Correspondence Address: FOLEY & LARDNER LLP, 1530 PAGE MILL ROAD, PALO ALTO, CA, 94304, US

(73) Assignee: The Regents of the University of California

(21) Appl. No.: 10/810,333

(22) Filed: Mar. 25, 2004

Related U.S. Application Data
(60) Provisional application No. 60/457,762, filed on Mar. 25, 2003.

(51) Int. Cl. C12Q 1/68; C12M 1/34

(52) U.S. Cl. 435/6; 435/287.2; 205/777.5

(57) ABSTRACT

A reagentless, reusable bioelectronic DNA, or other oligonucleotide sequence sensor is disclosed. The sensor includes an oligonucleotide probe tagged with a electroactive, redoxable moiety, self-assembled on or near an electrode. This surface-confined oligonucleotide probe structure undergoes hybridization-induced conformational change in the presence of the target oligonculeotide sequence which change the electron-transfer distance between the redoxable moiety and the electrode thereby providing a detectable signal change. In an alternative embodiment, the target can harbor the redoxable moiety. In a preferred application, the target sequence is associated with an object and its detection is correlated with the authenticity of the object.

### REFERENCE TO RELATED APPLICATION

This application is related to and claims the benefit of U.S. Provisional Application Ser. No. 60/457,762 filed on Mar. 25, 2003 and U.S. application Ser. No. 10/678,760 filed on Oct. 3, 2003.

### REFERENCE TO GOVERNMENT SUPPORT

This invention was made in part with government support under grants from the National Science Foundation (Grant No. NSF-DMR-0099843), the Office of Naval Research (Grant No. ONR N0014-1-1-0239) and the National Institute of Health (Grant No. GM 62958-01).

### BACKGROUND OF THE INVENTION

1. Field of the Invention

This invention relates to bioelectronic sensors and their use to detect hybridization events occurring in DNA, RNA, acids, PNA and other oligonucleotide systems. In a preferred embodiment the detection of such hybridization events is used to detect and verify an oligonucleotide authentication tag.

2. Background Information

The detection of DNA, RNA, nucleic acids comprising base analogs, thiols, etc., and, to a lesser extent, PNA (hereinafter generally referred to as “oligonucleotides” and/or “polynucleotides”) hybridization events is of significant scientific and technological importance, manifested in, for example, the rapidly growing interest in the chip-based characterization of gene expression patterns and the detection of pathogens in both clinical and civil defense settings [Heller, M. J., Annu. Rev. Biomed. Eng. 4, 129-153 (2002)]. Consequently, a variety of optical [Taton, T. A., Mirkin, C. A. & Letsinger, R. L. Science 289, 1757-1760 (2000); Gaylord, B. S., Heeger, A. J. & Bazan, G. C., Proc. Nat. Acad. Sci. USA 99, 10954 (2002); Cao, Y. W. C., Jin, R. C. & Mirkin, C. A., Science 297, 1536-1540 (2002)] and acoustic [Cooper, M. A. et al. Nature Biotech. 19, 833-837 (2001)] detection methods have been proposed.

In these assays one or more target oligonucleotides is brought into proximity to one or more oligonucleotide ligands and hybridization (if any) is detected by noting a change in a detectable “genosensor” moiety such as the presence of a suitable fluorolabel, radiolabel or enzyme label, present on the ligands.

Among these historic genosensors, fluorescence detection methods have historically dominated the state of the art [Heller, M. J., Annu. Rev. Biomed. Eng. 4, 129-153 (2002); Bowtell, D. D. L., Nature Genet. 21, 25-32 (1999); Winzeler, E. A., Schena, M. & Davis, R. W., Methods Enzymol. 306, 3 (1999)].

The application of electronic methods to the sensing of biologically related species has recently been attracting increased attention [Kuhr, W. G., Nature Biotech. 18, 1042-1043 (2000); Willner, I., Science 298, 2407 (2002); Fritz, J., Cooper, E. B., Gaudet, S., Sorger, P. K. & Manalis, S. R. Electronic detection of DNA by its intrinsic molecular charge. Proc. Natl. Acad. Sci., U.S.A. 99, 14142-14146 (2002)].

Advantages of bioelectronic detection include the following:
1. Electrochemical techniques offer the promise of sensitive, rapid and inexpensive screening [Bard, A. J. & Faulkner, L. R. Electrochemical Methods (John W. Willey & Sons, New York, 2001)].
2. Unlike fluorophores that quench or photo-bleach, typical electroactive labels are stable and relatively insensitive to their environment.
3. “Multi-color” labeling is possible by molecular design and synthesis that produce a “spectrum” of derivatives, each having a unique detectable electronic signal [Brazill, S. A., Kim, P. H. & Kuhr, W. G., Anal. Chem. 73, 4882-4890 (2001)].
4. The possibility of mass-production of bioelectronic detectors via the well-developed technical infrastructure of the microelectronics industry, renders electronic detection particularly compatible with microarray-based technologies.

oligonucleotides are typically electrochemically silent at moderate applied voltages [Palecek, E. & Jelen, F., Crit. Rev. Anal. Chem. 32, 261-270 (2002)]. The first sequence-selective electronic method for DNA detection was based on the electrochemical interrogation of redox-active intercolators that bind preferentially to double-stranded DNA (dsDNA) [Millan, K. M. & Mikkelsen, S. R., Anal. Chem. 65, 2317-2323 (1993)]. More recently, the sensitivity of this detection approach was improved via electrocatalytic amplification [Kelley, S. O., Boon, E. M., Barton, J. K. & Jackson, N. M. H., Nucleic Acids Res. 27, 4830-4837 (1999)].

In an attempt to reduce high background deriving from the inappropriate binding of hybridization indicators to single-stranded DNA (ssDNA), a “sandwich” type detector has been developed. This approach utilizes an electrode-attached ssDNA sequence that binds the target to the electrode and a second, redox-labeled ligand sequence complimentary to an adjacent sequence on the target [Ihara, T., Maruo, Y., Takenaka, S. & Takagi, M., Nucleic Acids Res. 24, 4273-4280 (1996); Yu, C. J. et al., J. Am. Chem. Soc. 123, 11155-11161 (2001); Umek, R. M. et al., J. Mol. Diag 3, 74-84 (2001)].

Mirkin and co-workers have developed an electronic DNA detection approach that has demonstrated high sensitivity and selectivity [Park, S. J., Taton, T. A. & Mirkin, C. A,. Science 295, 1503-1506 (2002)]. In this resistance-based method, a probe-captured target undergoes a second hybridization event with Au nanoparticle-labeled DNA strands. Subsequent catalytic deposition of silver onto the Au nanoparticles leads to electrical contact and a detectable decrease in the resistance between electrode pairs as an indicator of hybridization.

Despite this interest in electronic oligonucleotide detection, there has been little progress toward the important goal of creating a sensor that is simultaneously sensitive, selective and reagentless (e.g., a sensor obviating further treatment with either hybridization indicators or signaling molecules to yield a detectable indication of hybridization). The “reagentless” feature has been reported in the context of a conjugated polymer-based electrochemical DNA sensor [Korri-Youssoufi, H., Gamier, F., Srivastava, P., Godillot, P. & Yassar, A., J. Am. Chem. Soc. 119, 7388-7389 (1997)]. However, this sensor has only moderate sensitivity due to broad, weakly-defined redox, peaks.

More generally, while sensitivity of electronic oligonucleotide sensors of the prior art is impressive (ranging from 0.5 to 32 pM), no electronic sensors have been reported to meet the goal of fM sensitivity. The sensitive sensors require the addition of one or more exogenous reagents.

Recent, high profile examples ranging from geopolitical (e.g., forged documents purporting the solicitation of yellow-cake sales to Iraq) to the medical (e.g. the recent recall of approximately 100,000 bottles of potentially counterfeit Lipitor tablets) are indicative of the growing and increasingly complex risks associated with the counterfeiting of a wide range of documents and materials. Thus motivated, significant research has focused on the development of convenient-yet-unforgeable means of “authentifying” the provenance of documents, drugs and other materials related to medical, industrial, homeland or military security.

The use of DNA as an identifying label was first proposed by Philippe Labacq in U.S. Pat. No. 5,139,812 (issued Aug. 18, 1992). The approach works by concealing coded messages in DNA. Security is provided by the inherent sequence complexity of DNA (Clelland, C. T., Risca, V. and Bancroft, C. Nature 399, 533-534 (1999)).

Existing DNA-based authentication methods, however, have been limited to art, sports memorabilia and other high-value, low-volume applications. More widespread use of the approach has been limited by the cumbersome, time and reagent-intensive methods currently employed for the detection of low concentrations of a target DNA sequence in the presence of orders of magnitude larger background of masking DNA (Clelland, C. T., Risca, V. and Bancroft, C. Nature 399, 533-534 (1999); Cox, J. P. L. Analyst 126, 545-547 (2001)). Unfortunately, the technologies underlying counterfeiting generally keep pace with the technologies aimed at impeding such efforts. Thus, to date, no general, unbreakable means of “authenticating” documents, drugs and other high-volume materials has been reported.

It is the object of this invention to provide an electrochemical method for detecting specific sequences on target oligonucleotides, said method being simultaneously sensitive, selective, reagentless, and reusable. It is a further object to provide an electrochemical method for detecting an oligonucleotide-based (such as, for example, a DNA, RNA or peptide nucleic acid (PNA)-based) authentication tag.

### STATEMENT OF THE INVENTION

We have now discovered a detector and system for determining the presence of a target oligonucleotide having a target nucleotide sequence. The detector has an electrode capable of sensing redox events in a redoxable moiety and an immobilized oligonucleotide probe designed to hybridizes with a target nucleotide sequence. Either the probe (also referred to as the “sensor,” “sensor element,” or the like) or target (also referred to as the “tag,” authentification tag,” or the like) further comprises a redoxable moiety (also referred to a “redox moiety,” “redox(able) chemical moiety,” or the like).

In the case where the redoxable moiety is attached to the probe, such probe has a first configuration, in the absence of hybridization with the target oligonucleotide, which locates the redox moiety in a first position relative to the electrode. The probe has a second configuration in the presence of hybridization with the target oligonucleotide, which locates the redox moiety in a second position relative to the electrode. The first and second positions give rise to distinguishable redox events that are detectable by the electrode.

The first position may be closer to the electrode than the second position or vice versa.

In presently preferred embodiments, the probe is immobilized on the electrode.

In some preferred embodiments one or both of the first and second configurations may include a stem and hairpin (stem and loop) configuration with the stem immobilized on the electrode and with the redox moiety attached to the end of the oligonucleotide probe distal from the stem.

In the case where the redoxable moiety is attached to the target oligonucleotide, the probe, in the absence of hybridization with the target oligonucleotide, produces only a background redox signal or produces no signal. A signal is produced only when the probe hybridizes with the target oligonucleotide harboring the redoxable moiety, thereby bringing the redoxable moiety in sufficiently close proximity to the electrode to produce a measurable signal.

In a second aspect, this invention concerns a method for detecting the presence of a target oligonucleotide having a target nucleotide sequence in a sample. This method involves contacting the sample under oligonucleotide hybridization conditions with the detector just described and sensing redox events in the redox moiety in the presence of the sample and redox events with the detector in the absence of the sample and correlating similarity in redox events between the two sensings with the absence of the target oligonucleotide and a change in redox events with the presence of the target oligonucleotide.

In a third aspect this invention provides a rapid, reagentless, E-DNA process for convenient, secure and inexpensive authentication. The E-DNA approach unambiguously determines the provenance of materials via the sequence specific detection of nanogram quantities of a oligonucleotide-based authentication tag. A many-fold excess of non-cognate, “masking oligonucleotides,” which may be additionally present to thwart efforts to forge the authentication tag via cloning or sequencing, or may be present as a consequence of contamination or inherent in the materials to be authenticated, does not detectably alter the authentication signal. Using an inexpensive electrochemical workstation, robust authentication signals are obtained via, e.g., salt-water extraction of authentication tags from dried paper, dissolution of a solid forms of drugs, or from a sample of a liquid solution or suspension of a drug, all in about 10 minutes, and without further processing or the addition of exogenous reagents.

### DETAILED DESCRIPTION OF THE INVENTION

### BRIEF DESCRIPTION OF THE DRAWINGS

This invention will be further described with reference being made to the drawings in which:

FIG. 1 is a not-to-scale, semi-perspective diagram illustrating the mechanism by which a detector of this invention provides an indication of an oligonucleotide hybridization event. In this embodiment, the detector provides a decrease in signal as a measure of hybridization. FIG. 1A is a not-to-scale, elevational side view of the diagrammatic depiction of the hybridization event shown in FIG. 1.

FIG. 2 is a second, not-to-scale diagram illustrating the mechanism by which a second embodiment of the detector of the invention provides an increase in signal as a measure of hybridization.

FIG. 3 is a third, not-to-scale diagram illustrating a third mechanism by which a third embodiment of the detector of the invention provides an indication of hybridization.

FIG. 4 is a fourth, not-to-scale diagram illustrating two additional mechanisms by which additional embodiments of the detector of the invention provides an indication of hybridization.

FIG. 5A is a cyclic voltammogram for a gold electrode modified with the ferrocene tagged, stem-loop-forming oligonucleotide in a 1 M NaClO₄ solution, at a scan rate of 0.1 V/s. FIG. 5B demonstrates the relationship between the peak current and the scan rate.

FIG. 6A is a series of background-subtracted [Fan, C., Gillespie, B., Wang, G., Heeger, A. J. and Plaxco, K. W., J Phys. Chem. (B) 106, 11375-11383 (2002); Hirst, J. et al. J. Am. Chem. Soc. 120, 7085-7094 (1998)] voltammograms (anodic scan) for a hairpin-forming, DNA-modified gold electrode in the presence of complementary DNA (cDNA) at different concentrations: 0, 30 pM, 500 pM, 30 nM, 800 nM, 5 μM (from bottom to top). The hybridization was performed in a 1 M NaClO₄ solution, and the hybridization time was fixed at 30 min. FIG. 6b is a calibration curve (peak height vs. concentration of the cDNA).

FIG. 7 is a graph illustrating that at a target DNA concentration of 500 pM, the signal develops in minutes. At this target DNA concentration, about 65% of the probe-DNA has been hybridized in one hour (at 5 mM target DNA, the signal goes to zero within 30 minutes).

FIG. 8 is a cyclic voltammogram for a gold electrode modified with a methylene blue-tagged oligonucleotide in the absence of target oligonucleotide.

FIG. 9 is a series of AC voltammograms for the E-DNA sensor before a test (upper line) and after a test with DNA microdots containing masking DNA only (lower line) and masking DNA with target DNA (upper line).

FIG. 10 is a graphic comparison among the E-DNA authentication signals observed before and after counterfeiting tests on three possible counterfitted objects.

FIG. 11 is a graphic comparison among E-DNA authentication signals generated in essentially the same manner as the signals in FIG. 10 with the addition of glycerol as an additive to reduce background noise. This figure displays the amount of signal change that was observed.

### DESCRIPTION OF PREFERRED EMBODIMENTS

Representative E-DNA Sensors

The present invention describes a reagentless, reusable method for detecting the presence and/or identity of oligonucleotides or polynucleotides using electronic sensors. The electronic sensors of the invention, which can be exquisitely sensitive, may be referred to herein as “E-DNA sensors.”

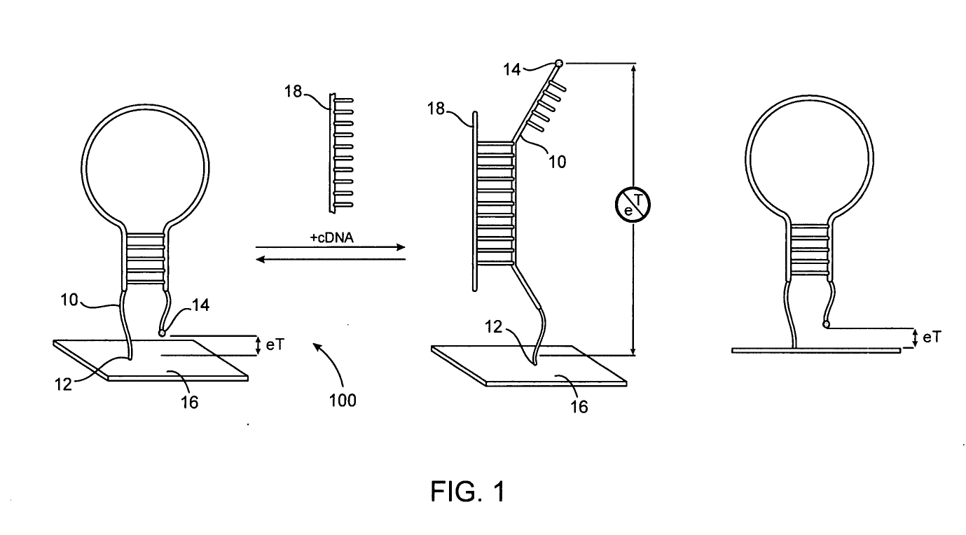

As shown in FIG. 1, the E-DNA sensors can employ oligonucleotides capable of forming “stem-loop” or “hairpin” structures (also referred to “stem-loop” or “hairpin” DNA, or simply “stem-loops” or “hairpins”), with an electroactive label to detect hybridization events. Stem-loop DNA is an extremely interesting structure that forms the basis of the fluorescent, “molecular beacon” approach for homogeneous, optical hybridization detection [Tyagi, S and Kramer, F. R., Nat. Biotechnol. 14, 303-308 (1996)]. In stem-loop DNA, the base sequence is designed such that the structure is initially in the folded “hairpin” configuration. Formation of the hairpin structure is precluded, or disfavored in terms of assay equilibria, following hybridization with its specific complementary base sequences. The existence of the stem-loop structure in the design provides an on/off switch as well as a stringency test sufficient to discriminate single-base mismatches.

As used herein, different stem-loop or hairpin structures, such as those that exist in the presence or absence of a target oligonucleotide or polynucleotide, may be referred to a “configurations”.

“Target” refers to an oligonucleotide or polynucleotide having a sequence to which a particular electroactively-labeled oligonucleotide (also referred to as a “sensor” or “probe”) is designed to hybridize.

The particular use of terms “oligonucleotide” and “polynucleotide” should in no way be considered limiting. “Oligonucleotide” is used when the relevant nucleic acid molecules typically comprise less than about 100 bases. “Polynucleotide” is used when the relevant nucleic acid molecules typically comprise more than about 100 bases. Both terms are used to denote DNA, RNA, modified or synthetic DNA or RNA (including but not limited to nucleic acids comprising synthetic and naturally-occurring base analogs, dideoxy or other sugars, and thiols), and PNA or other nucleobase containing polymers. However, probes and/or targets may comprise fewer than or more than 100 bases (inclusive). Accordingly, the terms “oligonucleotide” and “polynucleotide” are used to describe particular embodiments of the invention. The terms in no way define or limit the length of the nucleic acids that may be used to practice the invention.

In sensor 100 of FIG. 1, a hairpin oligonucleotide 10 possessing, for example, a thiol 12 and a redoxable chemical moiety 14 such as, for example, a ferrocene group or a methylene blue group, is immobilized on a gold electrode 16 via self-assembly. In the “closed state,” oligonucleotide 10 presents a stem-loop structure that localizes the redoxable chemical moiety 14 in close proximity to the gold surface 16. Thus the distance between the gold and redoxable chemical moiety is sufficiently short for facile electron transduction (eT), thereby enabling redox of the redoxable chemical moiety in response to potentials applied via electrode 16. In the “open state,” following hybridization with a complementary oligonucleotide 18, electron transfer between the redoxable chemical moiety 14 and the electrode 16 is blocked since moiety 14 is separated from the electrode surface. The distance for which eT is or is not facile is clearly illustrated in FIG. 1A, which corresponds to FIG. 1 but is drawn in side view, as opposed to FIG. 1, which is in semi-perspective view.

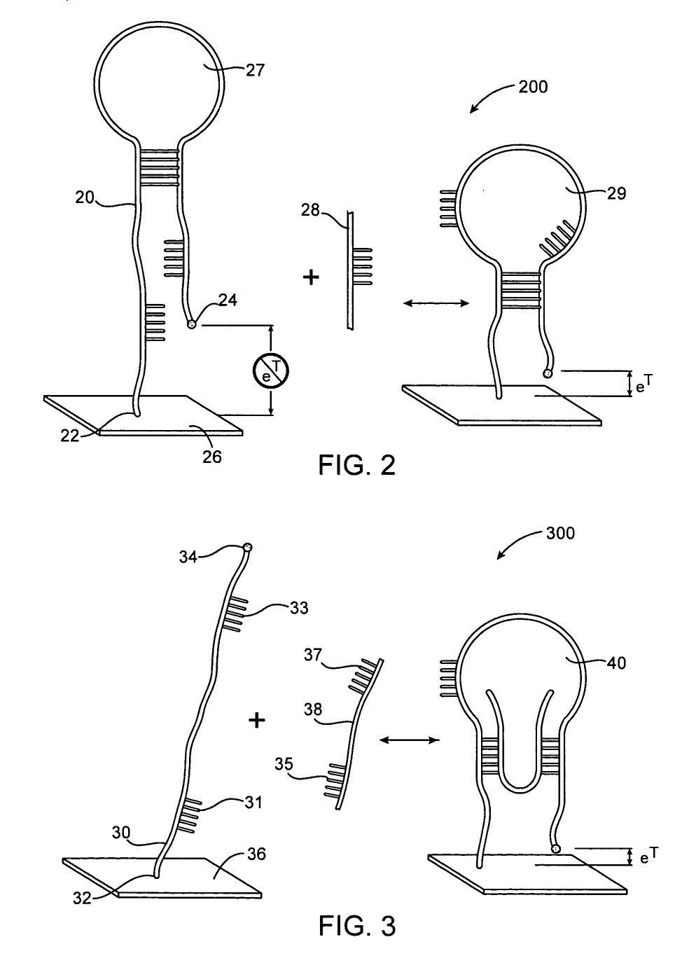

In the embodiment 100 described in FIG. 1, the E-DNA sensor suffers from being a “signal-off” sensor. That is, in response to its target, the electrochemical signal is abolished. This renders that embodiment of the E-DNA detector vulnerable to false positives arising via disruption of the stem-loop sensor element by environmental conditions or physical degradation (e.g. by nucleases). As shown in FIG. 2, with the appropriate oligonucleotide design, a “signal-on” E-DNA sensor 200 can be engineered, thus silencing false positives arising due to chemical or enzymatic destruction of the sensor element. The appropriate structure contains an oligonucleotide probe 20 attached to or adjacent to electrode 26 at end 22. The other end of probe 20 carriers a redoxable moiety 24. In one configuration, probe 20 contains a moderate length hairpin 27 that positions the electroactive label 24 away from the electrode 26. That hairpin configuration 27 thermodynamically competes with a less stable hairpin configuration 29. The less stable hairpin 29 positions the label 24 in proximity to the electrode 26. Hybridization with target 28 disrupts and/or destabilizes hairpin structure 27, favoring the formation of hairpin structure 29, which brings the label 24 into proximity with the electrode 26, resulting in a signal.

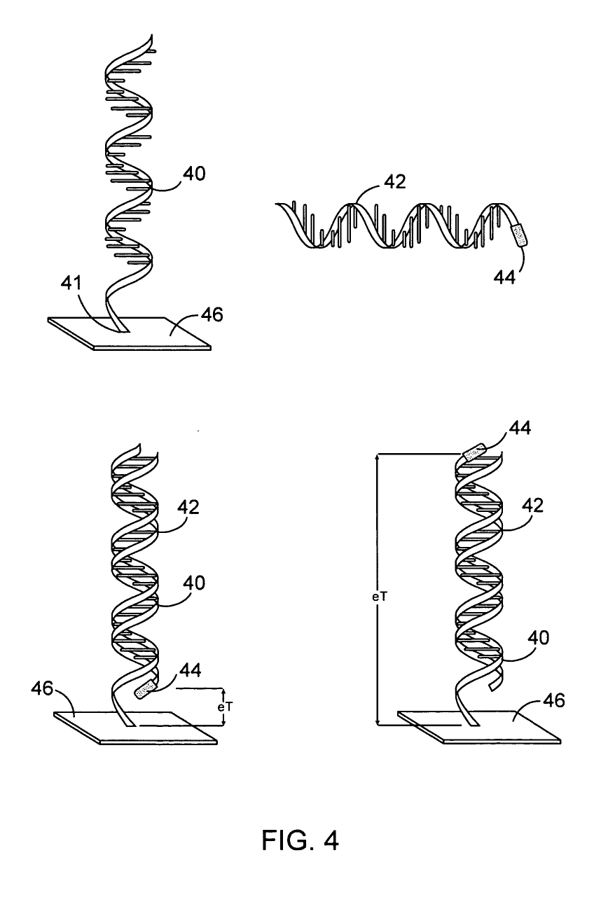

In another embodiment, as shown in FIG. 3, an oligonucleotide probe 30 may be coupled near or to electrode 36 via bond 32. The end of probe 30 distant from the point of attachment 32 is labeled with redoxable moiety 34. In the absence of target 38, probe 30 is “open” and label 34 is a long distance from electrode 36. In this embodiment, probe 30 contains regions 31 and 33 which are complementary to regions 35 and 37 on target 38. When target 38 and probe 30 are hybridized, target 38 bridges regions 31 and 33 of probe 30 to form loop 40, and thus positions redoxable moiety 34 in sufficient proximity to electrode 36 to promote electron transduction, which can be detected.

As shown in FIG. 4, one can also achieve a signal based on hybridization in systems not involving stem-loop or hairpin structures. In FIG. 4, an oligonucleotide 40 possessing a terminal thiol group or other suitable binding group is immobilized at a gold electrode 46 via bond 41. A target 42 bearing redoxable label 44 is brought into proximity to the bound oligonucleotide 40. In the absence of target there is no signal. Upon hybridization with the target, the label is brought into sufficient proximity to the electrode to allow electron transfer (eT), producing a measurable electrochemical signal.

In this embodiment, the hybridization system of the invention utilizes an electrochemical approach with a “signal-on” feature to identify oligonucleotide tags. The strategy demonstrated in FIG. 4 involves a gold electrode 46 and a DNA probe strand 40 without electroactive labels. The probe 40 sequence is designed to be complementary to the target oligonucleotide or polynucleotide 42 (e.g., an authentication tag present in a material to be authenticated) and contains a 5′ thiol. The probe is assembled on the gold surface through gold-thiol chemistry. The target 42 additionally comprises methylene blue as the electroactive label 44 at either its 5′ end, its 3′ end, or both. The target may be encapsulated or otherwise secreted in documents or drugs. Prior to detection, the gold electrode has no signal since it has only the probe DNA without a redoxable moiety. After hybridization with target oligonucleotides (e.g., eluted from material to be authenticated) the label is brought to the electrode surface and creates the electrochemical signal. The “signal-on” process is due to either the direct electron tunneling into the redox molecule from the gold electrode (left image), or through electron transfer mediated by the hybridized DNA (right image) (Boon, E. M., Salas, J. E. and Barton, J. K., Nat Biotechnol. 20, 282-286 (2002)). Since the signal is created only after hybridization, this approach offers the advantage of being insensitive to environmental contaminants.

These are but four representative configurations for the E-DNA sensor. Any probe configuration which will present different configurations in the presence and absence of target DNA, and that can reposition a redox label in electrically distinguishable different proximities to the sensing electrode can be used.

Representative Materials

In the embodiments just described, the redoxable chemical moiety has been ferrocene or methylene blue. More generally, any redoxable chemical moiety that is stable under assay conditions can be used. Examples include, but are not limited to, purely organic redox labels, such as viologen, anthraquinone, ethidium bromide, daunomycin, methylene blue, and their derivatives, organo-metallic redox labels, such as ferrocene, ruthenium, bis-pyridine, tris-pyridine, bis-imidizole, and their derivatives, and biological redox labels, such as cytochrome c, plastocyanin, and cytochrome c′.

In a preferred embodiment, the electrode is fabricated from known electrode materials such as, for example, gold, silver, platinum, carbon, or silicon. Gold gives good results.

In a preferred embodiment, the surface of the electrode is functionalized with the olilgonucleotide probe structure through self-assembly, such as through the well-established gold-S chemistry of self assembly.

It is also preferred that when the electrode surface, functionalized with the oligonucleotide probe structure, is subsequently passivated by materials such as 2-mercaptoethanol, (2-ME), 6-mercaphohexanol or mercaptoalkanols generally (HS—(CH₂)ₙ—OH with n=2˜18) and the like.

Ideally, the stem-loop oligonucleotide structures are loosely packed on the gold surface in order to minimize steric effects that could interfere with hybridization.

Preferred embodiments for stem-loop structure are well known in the art. For example, the stem-loop structure may be designed such that about five bases at a relative 5′-end and relative 3′-end are fully complementary. The base sequence in the loop region of the stem loop DNA may be selected so as to be complementary to the specific base sequence to be detected in the target DNA. In addition, the use of complementary G-C rich sequences may be desirable to enhance “stem” stability in stem-loop structures.

In some embodiments, the probe structure comprises an oligomer of neutral peptide nucleic acid (PNA) in place of the DNA oligonuceotide to allow hybridization to occur at ambient ionic strengths. In addition to silencing and detecting false positives, degradation of the sensor element can be avoided by building the stem-loop element from peptide nucleic acid (PNA). PNA is chemically and enzymatically robust and, because it is uncharged, forms stronger duplexes with DNA or RNA than ssDNA. Thus, there are clear advantages to “E-DNA” sensors comprising synthesized PNA sensor elements.

The oligonucleotide probe may be attached to the electrode via a “molecular-wire” such as, for example, an oligo(phenylene vinylene) in order to facilitate electron transfer.

The sensor can also employ aptemers. An aspect of the E-DNA detection is the electrochemical detection of a target-induced conformational change. This means that this invention may be generalizable to other types of tags and analytes where conformational change occurs upon binding, such as protein folding or aptemer folding based biosensors.

Aptemers are DNA or RNA molecules that adopt well-defined tertiary structures analogous to natural enzymes. Aptemers have emerged as promising therapeutic and diagnostic tools [Chang, K. Y. & Varani, G., Nature Struct. Biol. 4, 854-858 (1997); Burgstaller, P., Girod, A. & Blind, M., Drug Discov. Today 7, 1221-1228 (2002); Wilson, D. S. & Szostak, J. W., Annu. Rev. Biochem. 68, 611-647 (1999)]. Well-developed in vitro selection methods have been able to produce aptemers for virtually any target [Wilson, D. S. & Szostak, J. W., Annu. Rev. Biochem. 68, 611-647 (1999); Griffiths, A. D. & Tawfik, D. S., Curr. Opin. Biotech. 11, 338-353 (2000)]. Given these advantages, oligonucleotide aptemers are anticipated to play an important role in next-generation biosensing elements [Sullivan, C. K. O., Anal. Bioanal. Chem. 372, 44-48 (2002); Robertson, M. P. & Ellington, A., Nature Biotech. 17, 62-66 (1999)].

DNA or RNA aptemers that undergo significant conformational changes upon binding specific analytes are readily available. In vitro selection techniques are able to isolate highly affinitive RNA or DNA aptemers that bind almost any arbitrary small molecule, biomacromolecule or cell type. Many aptemers undergo significant conformational changes upon analyte binding. Alternatively, although insignificant signal changes are expected for aptemers that undergo subtle conformational changes, it is feasible to accomplish analyte detection via combining a recently proposed aptemer self-assembly approach [Stojanovic, M. N., de Prada, P. & Landry, D. W., J. Am. Chem. Soc. 122, 11547-11548 (2000)]. For example, aptemers rationally dissected into two halves, with one immobilized at electrode surfaces and the other tagged with electroactive label, are expected to be split in the absence of analytes while self-assembled upon analyte binding. Thus the approach described here can be generalized from stem-loop structures to DNA and RNA aptemers and thereby to sensing platforms directed against essentially any water soluble analyte.

Reaction Conditions and Detection Methods

The hybridization events that are sensed by the detectors, and methods of this invention, are carried out in aqueous liquid environment. Aqueous environments are preferable but optionally rendered at least somewhat ionic by the presence of dissolved salt. It is generally understood that ionic environments favor hybridization. “Salt” is defined to include sodium chloride but also any other water-soluble alkaline earth or alkyl metal ionic materials. Magnesium, potassium, calcium, and/or manganese salts may be particularly useful for practicing the invention. While there may be advantages to particular salt materials or levels, they are not seen to be critical to the practice of this invention. Representative salt levels can be as high as about 4 or 5 molar, in some cases and as low as nearly zero. In the examples, 1 molar NaCl is generally used. Thus, salt levels of from about 0.05 to about 2 molar are presently preferred. In a particular embodiment of the invention, a physiological salt concentration (i.e., about 150 mM) is used. In other embodiments of the invention, the salt concentrations may bracket physiological salt conditions, e.g., from about 75 mM to about 300 mM.

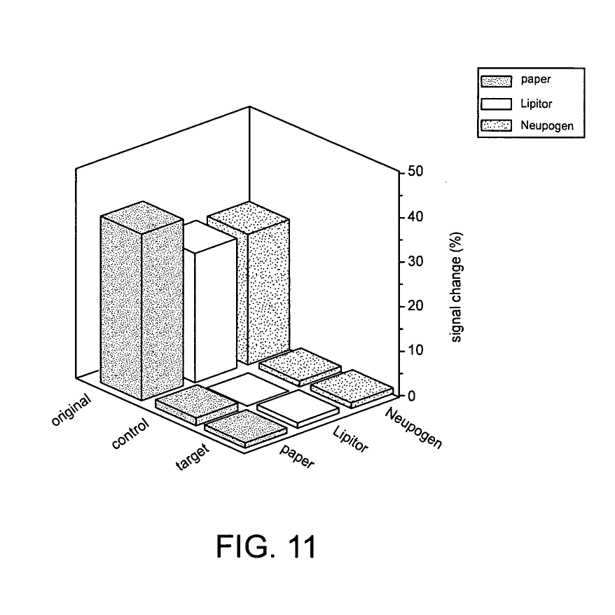

Hybridization can be carried out in the presence of agents and additives that promote the desired hybridization, diminish nonspecific background interactions, inhibit the growth of microorganisms, or increase the stability of the probe and/or target oligonucleotides. For example, one can add up to 10% by weight or volume (based on the amount of aqueous environment) and particularly from about 1 or 2% to about 10% of one or more polyols. Representative polyols include glycerol, ethylene glycol propylene glycol sugars such as sucrose or glucose, and the like. One can also add similar levels of water soluble or water dispersible polymers such as polyethylene glycol (PEG) or polyvinyl alcohol or the like. Another representative additive is up to about 1 or 2% by weight (again based on the liquid substrate) of one or more surfactants such as triton X-100 or sodium dodecyl sulfate (SDS). All of these agents are electrochemically silent at the potentials observed with the sensors and methods of the invention. As a comparison of the results shown in FIG. 11 with the results shown in FIG. 10 make clear, the use of certain additives can lead to dramatic improvements in signal. A variety of hybridization conditions have been described and are well known in the art. Many such hybridization conditions are useful for practicing the invention.

Hybridization can be carried out at ambient temperature, although any temperature in the range over which hybridization is stable can be used. A preferred range is from about 5 to about 45° C. Hybridization times should be a short as possible for convenience. Times as short as minutes (e.g., about 1 to 5 minutes) can be used. Times of up to 5, 10, 15, 20, 30, 45, or 60 minutes, or longer may also be used. We have had good results with hybridization times of from about 15 to about 45 minutes. Hybridization temperatures and times may be determined empirically or using, e.g., CoT analysis or other methods of predicting hybridization conditions.

Multiplexing

False positives can be identified via multiplexing, e.g., using multiple, electrochemically distinct labels, such that the sensor and one or more control elements are integrated into a single sensor pixel. By employing multiple labels with narrow, non-overlapping redox potentials, 2-5 or possible more distinct sequences can be simultaneously interrogated on a single electrode. This enables the inclusion of internal controls, i.e., elements that are not complementary to known sequences that would respond to false positives arising due to non-specific disruption or degradation of the stem-loop. Multiplexing will also facilitate signal redundancy, alleviating the risk of masking in the unlikely event of contaminants with redox potentials precisely where the primary label reports. In addition to exhibiting narrow, non-overlapping redox peaks, the appropriate labels for multiplexing should be stable and synthetically facile.

Electroactive labels that meet these requirements, include a large number of ferrocene [Brazill, S. A., Kim, P. H. & Kuhr, W. G., Anal. Chem. 73, 4882-4890 (2001)] and viologen derivatives (Fan, C., Hirasa, T., Plaxco, K. W. and Heeger, A. J. (2003)) Langmuir, and any redoxable species, such as methylene blue, anthraquinone, ethidium bromide, daunomycin.

Improving Sensitivity

AC voltammetric methods are commonly employed in an effort to delineate between redox and charging currents based on the different timescales for the two processes. Double-layer formation is limited only by ion mobility and thus equilibrates rapidly, whereas redox currents are limited by Marcus-type barriers and is orders of magnitude slower. Sinusoidal voltammetry (SV) or pulsed voltammetry has proven particularly useful; in addition to the SV frequency spectrum, time course data is obtained at each harmonic frequency element by performing the digital equivalent of a lock-in amplifier (Brazill S A, Bender S E, Hebert N E, et al. J. Electroanal. Chem., 531, 119-132 (2002)). That is, the instantaneous current is monitored at the optimum phase angle for the signal of interest, thus greatly increasing the sensitivity and selectivity over traditional voltammetric techniques. This temporal deconvolution enables a large increase in peak to charging current ratios and thus an improvement in the E-DNA sensitivity by orders of magnitude. Cyclic voltammetry is also used.

Improving Peak Currents

The use of multiple redoxable chemical moieties will significantly increase the sensitivity. A straightforward approach to this end would be to label the single sensor strand with multiple redoxable chemical moieties. The sensor oligonucleotide element in FIG. 1 is modified on the 2′ position of the terminal nucleotide, but modification of internal nucleotides is equally facile and should not significantly reduce the stability of the stem element. Because the electroactive label is isolated from the nucleotide, e.g., by a pentyl linker, the labels will not interact with one another and thus multi-labeled sensor elements will exhibit redox peaks at the same potential (and peak width) as single-labeled probes. Because peak current is proportional to the number of electron acceptors/donors this approach will only improve peak currents by a factor of 2-5, with the upper limit corresponding to the number of electron acceptors that can be packed onto the about 5 bases in the terminal stem sequence.

Electrocatalysis, in contrast, provides a potential means of increasing peak currents by orders of magnitude. The approach works by the addition of an electrochemical mediator, such as ferrocyanide, that is not reduced by the electrode but can be reduced by the ferrocene label (Boon, E. M., Ceres, D. M., Drummond, T. G., Hill, M. G., Barton, J. K. Nat. Biotech., 18, 1096-1100 (2000)). Thus, in the presence of ferrocyanide, the electrode repeatedly reduces each ferrocene, thus catalytically increasing peak currents. This approach leads to a sensor that is no longer reagentless.

Tag Detection and Authentication

This invention provides a reagentless, electronic means for rapidly, specifically and inexpensively detecting DNA-based authentication tags, optionally in the presence of security-relevant levels of masking DNA (discussed in more detail, below). The method is suitable for the authentication of a wide range of items, including but not limited to, documents, medications (e.g., ingested, inhaled, injected, and/or topical pharmaceutical agents), food, and any physical objects in or upon which an oligonucleotide can be associated for the purpose of later authentification.

With this E-DNA sensor and optionally alternating current voltammetry (ACV), it is possible to produce a read-out of information encoded in an oligonucleotide, whether it be on packaging or on a label or the like or deposited on or dispersed in a solid or liquid. Only a small amount of oligonucleotide (e.g., about 5 ng for paper and about 20 ng for drugs) is necessary for detection as an authentication tag. More importantly, the E-DNA sensor can discriminate against a great excess of non-cognate oligonucleotide, which acts as a mask in order to thwart efforts to forge the authentication tag via cloning or sequencing.

Accordingly, oligonucleotides the E-DNA sensor conveniently identifies the hidden oligonucleotide sequence information in minutes. Given the simplicity and usefulness of this novel technology, it finds application in a variety of markets.

The one or more oligonucleotide authentification tags that will take part in the hybridization to a probe sequence may be mixed with a multi-fold concentration, such as 50 fold to 500,000 fold, e.g. 10,000 fold of non-cognate, masking oligonucleotide, and used in document and drug authentication and the like.

In this application, the oligonucleotide solution containing both authentication tag and masking oligonucleotide may be deposited on a piece of paper or similar carrier material. The paper is dried and associated with (e.g., attached to or the like) to the object to be authenticated. In the authentication stage, the paper is immersed in, for example, salt water to elute the tag. The eluted tag is ready for E-DNA detection.

In similar embodiments, oligonucleotide tags may be admixed in a solid, for example, a solid drug such as Lipitor powder, and thereafter dispersed in salt water and tested by the E-DNA sensor.

The E-DNA authentication strategy is particularly refractory to counterfeiting. The extremely high selectivity of the E-DNA sensor enables us to specifically detect the authentication oligonucleotide sequence even in the presence of up to about a 10,000-fold, or even greater, excess of non-cognate “masking” oligonucleotide. Representative masking levels can be a three-fold or greater excess, preferably a ten-fold or greater excess, and especially a 50-fold or greater excess. This high level of masking would render it extremely difficult to forge the authentication tag via cloning or other amplification, sequencing and/or copying methods. Moreover, the E-DNA approach is also suitable for the detection of peptide nucleic acid (PNA)-based authentication tags. Because PNA cannot be amplified or sequenced via enzymatic methods the use of such tags would render the approach still more refractory to copying-based counterfeiting.

As described, the E-DNA approach could potentially be partially circumvented via dilution, i.e., the extraction and dilution of the authentication tag from one document and its application to several forged documents, or via the inclusion of materials (denaturants, nucleases, etc.) that would disrupt or overwhelm stem hybridization. Attacks based on the former, however, can be frustrated via measurements of the absolute oligonucleotide concentration in the authentication sample and ratiometric measurements of the absolute oligonucleotide content versus the concentration of authentication tag. Methods of quantitating nucleic acids are well-known in the art. Similarly, the latter circumvention can be thwarted by ratiometric measurements of authentication tag versus control sequences known to be absent in authentic goods. Given that the small electrode size and reagentless nature of the E-DNA sensor renders it particularly well suited for dense, electronic sensor arrays, such radiometric measurements do not present a significant hurdle.

Microelectrodes and Arrays:

Because E-DNA involves an electronic sensor (e.g., a redoxable moiety), advances in electrophoretically-improved hybridization times can be applied [Cheng, J., Shoffner, M. A., Hvichia, G. E., Kricka, L. J., Wilding, P. (1996). Nuc. Acid Res., 22, 380-385; Cheng, J., Sheldon, E. L., Wu. L., Uribe, A., Gerrue L. O, Heller, M. O'Connell, J. (1998). Nat. Biotech. 16, 541-546]. Moreover, because of its direct integration into electronics and excellent scalability (in the Example 1, 2 mm² electrodes were used, but E-DNA's impressive signal strength suggests that significantly smaller electrodes can be employed), E-DNA is well suited for applications in electronic gene detection arrays. To this end, biomaterials can be deposited onto specific pixels of gold “nanode” arrays and electrochemically addressed.

In a more preferred embodiment, the microelectrodes are arrayed in the format of N “pixels” with each pixel containing a unique stem-loop or the like oligonucleotide structure and with all microelectrodes electrochemically addressable, thereby enabling detection of N different targets.

As demonstrated in the following examples, the bioelectronic sensor described herein is both sensitive and highly selective. The sensitivity and selectivity of the E-DNA sensor is better than that of typical CCD-based fluorescent detectors, and is comparable to a recently proposed, conjugated polymer-based fluorescence amplification method [Gaylord, B. S., Heeger, A. J. and Bazan, G. C., Proc. Nat. Acad. Sci. U.S.A. 99, 10954 (2002); Moon, J. H., Deans, R., Krueger, E. and Hancock, L. F., Chem. Commun., 104-105 (2003)]. The key sensing element, e.g., an oligonucleotide associated with a rexobale moiety, is compatible with normal solid-state synthesis of oligonucleotides. Moreover, the surface assembly process is robust and facile. Since the entire set-up can be conveniently prepared and is generally compatible with chip-based technology, the novel, reagentless detection described here provides a promising alternative to fluorescence-based sensors for most if not all of their applications.

The following general methods and specific examples are presented to illustrate the invention and are not to be considered as limitations thereon.

### EXAMPLES

### Example 1

### Fabrication of the Stem-Loop DNA Structure

Ferrocene carboxylic acid was purchased from Aldrich (Milwaukee, Wis.), 1-Ethyl-3-(3-dimethylaminopropyl)-carbodiimide (EDC) and N-hydrosuccinimide ester (NHS) were obtained from Sigma (Milwaukee, Wis.). Ferrocene succinimide ester (Fc-NHS) was prepared as described in the literature [Takenaka, S., Uto, Y., Kondo, H., Ihara, T. & Takagi, M. Anal. Biochem. 218, 436 (1994)] and confirmed by ¹H NMR. Oligonucleotides were obtained from Synthegen (Houston Tex.). The sensor oligonucleotide, sequence 5′-NH₂—(CH₂)₆-GCGAG GTA AAA CGA CGG CCA GT CTCGC-(CH₂)₆—SH-3′ (oligo 1), contained a 5′ hexamethylene amine and a 3′ hexamethylene thiol group. Fc-NHS was dissolved in a small volume of dimethyl sulfoxide and then diluted in a 0.1 M Na₂CO₃ buffer (pH 8.5) containing 0.1 mM of oligo 1. This mixture was stirred overnight at room temperature. The final product (oligo 1-Fc) was purified by HPLC on a C18 column and confirmed by electrospray mass spectroscopy. The sequences of the target and control DNA oligos were 5′-ttttt ACT GGC CGT CGT TTT AC tcttt-3′ and 5′-CGT ATC ATT GGA CTG GCC ATT TAT-3′. All solutions were prepared with nano-pure water.

### Example 2

### Preparation of the Functionalized Au Electrode

Polycrystalline gold disks (1.6 mm diameter; BAS Inc., West Lafayette, Ind.) were used as working electrodes. The protocol for gold electrode preparation has been previously described [Fan, C., Gillespie, B., Wang, G., Heeger, A. J. & Plaxco, K. W., J Phys. Chem. (B) 106, 11375-11383 (2002)]. The cleaned gold electrode was rinsed, dried under argon and then immediately incubated overnight in 1 M oligo 1-Fc, 10 mM phosphate buffer with 0.1 M NaCl, pH 7.4. Prior to use, the oligo 1-Fc was pre-treated with tris-(2-carboxyethyl)phosphine to break disulfide bonds and then purified using a spin column. The modified electrode was washed with water, dried under argon and incubated in 1 M NaClO₄ solution prior to use.

The gold surface was then functionalized by oligo 1 (see Example 1) through the well-established gold-S chemistry of self-assembly. Previous studies have demonstrated that this self-assembly process is only feasible in the presence of salt; in that high ionic strength leads to high surface density and closely packed DNA strands while low ionic strength produces loosely packed DNA strands [Boon, E. M., Salas, J. E. & Barton, J. K., Nature Biotech. 20, 282-286 (2002)]. For this Example, a relatively low ionic strength (0.1 M NaCl) was chosen to produce a loosely packed surface in order to minimize steric effects that could interfere with reversible hairpin formation (see FIG. 1). The prepared surface was subsequently passivated by 2-mercaptoethanol (2-ME). This process has been reported to “cure” the relatively disordered self-assembled monolayer (SAM) by gradually displacing nonspecifically adsorbed oligonucleotides [Heme, T. M. & Tarlov, M. J., J. Am. Chem. Soc. 119, 8916-8920 (1997)]. This oligonucleotide-containing, passivated surface has proven to be resistant to random DNA sequences, as reported previously [Heme, T. M. & Tarlov, M. J., J. Am. Chem. Soc. 119, 8916-8920 (1997)] and independently confirmed in our labs by monitoring with a quartz crystal microbalance.

### Example 3

### Characterization of the E-DNA Modified Electrode

The stem-loop structure localizes the ferrocene tag in close proximity to the gold surface (see Example 2 and FIG. 1) and thereby ensures that the distance between the gold electrode and the ferrocene moiety is sufficiently short to promote electron transfer.

Cyclic Voltammetry (CV) was performed using a CHI 603 workstation (CH Instruments) combined with a BAS C-3 stand. A platinum electrode was used as a pseudo-reference electrode while potentials are reported versus the normal hydrogen electrode (NHE). Background subtraction was conducted in some cases using Origin 6.0 (Microcal Software, Inc.) in order to remove non-Faradayic currents and improve signal clarity [Fan, C., Gillespie, B., Wang, G., Heeger, A. J. & Plaxco, K. W., J Phys. Chem. (B) 106, 11375-11383 (2002); Hirst, J. et al. J. Am. Chem. Soc. 120, 7085-7094 (1998). Bard, A. J. & Faulkner, L. R. Electrochemical Methods (John W. Willey & Sons, New York, 2001)]. All experiments were conducted at room temperature.

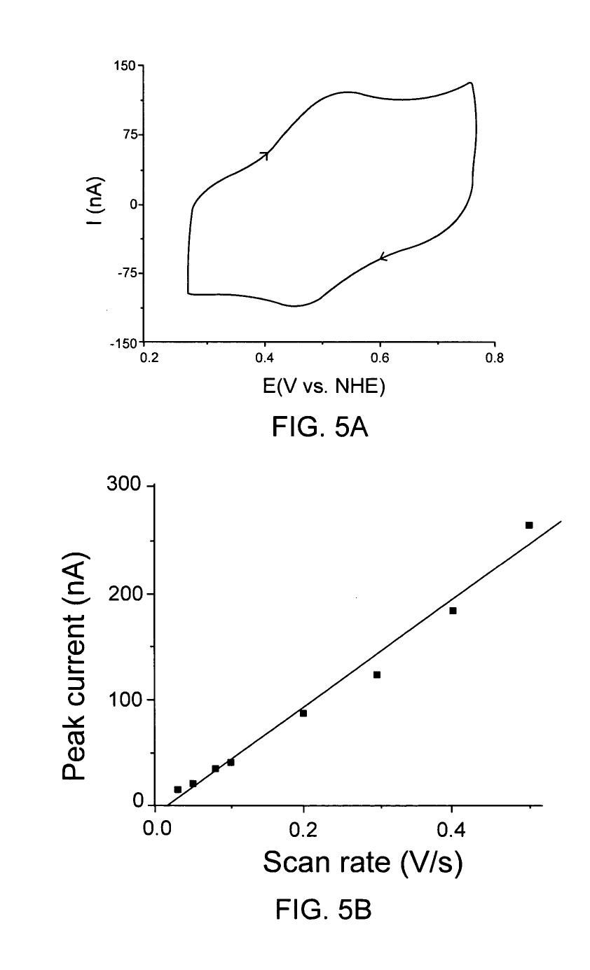

In the absence of target DNA, ferrocene redox peaks were observed (FIG. 5a). For comparison, a bare gold electrode or gold modified with either 2-ME or 2-ME/mercapto-oligonucleotides lacking ferrocene produces featureless CV curves in the same potential window. The apparent formal potential of the electroactive label is E⁰=0.492 V, as estimated from E_{1/2}=(E_{red}+Eₒₓ)/2. This value falls within the typical redox potential range of ferrocene (E⁰ of ferrocene is slightly sensitive to the environment, but remains within a relatively limited potential range) [Brazill, S. A., Kim, P. H. & Kuhr, W. G., Anal. Chem. 73, 4882-4890 (2001)]. Therefore, this peak pair was ascribed to the redox conversion of ferrocene labels in close proximity to the gold electrode. It is known that high salt concentration is required for the formation of short stem-loop structures as a result of the electrostatic repulsion between negatively charged DNA chains [Herne, T. M. & Tarlov, M. J., J. Am. Chem. Soc. 119, 8916-8920 (1997)]. We found that some freshly modified electrodes do not produce redox peaks without prior incubation in 1 M NaClO₄. This result provided strong evidence that the formation of the stem-loop structure facilitated the electron transfer between the gold electrode and ferrocene by constraining the ferrocene label in close proximity to the electrode surface. This result also implied that the use of neutral peptide nucleic acids (PNA) in place of the DNA might provide significant advantages by allowing hybridization to occur at ambient ionic strengths.

Modulating the scan rate of the CVs provided further evidence that ferrocene was confined at the electrode surface by the formation of the stem-loop structure. Peak currents of the ferrocene redox reaction (Ip) were directly proportional to scan rates (FIG. 5b), consistent with a surface-confined electrochemical reaction (in contrast to Ip being proportional to the square-root of the scan rate characteristic of diffusion-controlled electrochemical reactions) [Bard, A. J. & Faulkner, L. R. Electrochemical Methods (John W. Willey & Sons, New York, 2001)].

### Example 4

### Target DNA Detection

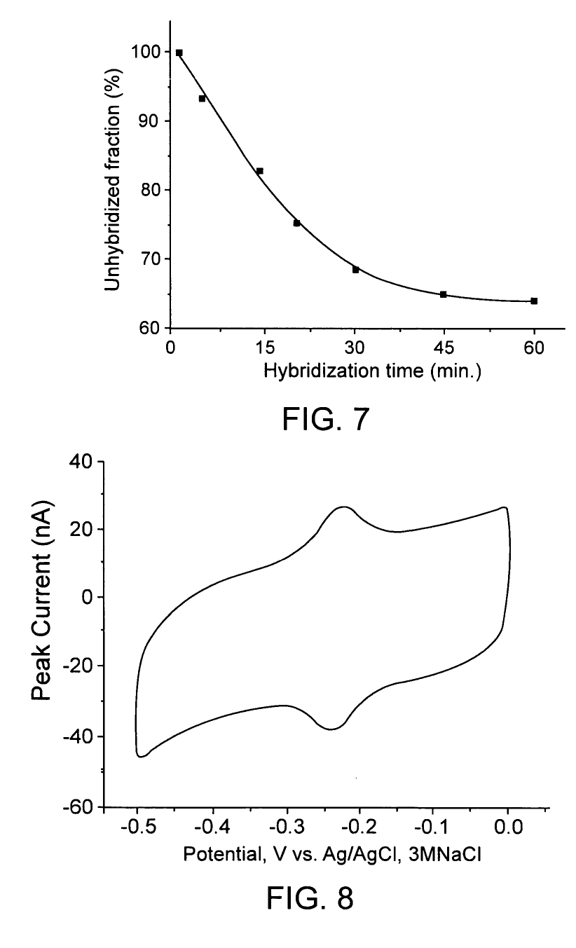

When the stem-loop structure encounters a sequence complementary to the loop region (17 bases), hybridization disrupts the less stable stem structure and isolates the ferrocene from the electrode surface. Thus, incubating a stem loop-modified electrode in a 5 M cDNA (oligo 2, see Example 1) solution containing 1 M NaClO₄ eliminated the ferrocene reduction and oxidation peaks within 30 min (FIG. 5a). After incubating the electrode with 500 pM cDNA solution and monitoring the hybridization process electrochemically, we observed that the electrochemical signal attenuated with a time constant of approximately 30 min (FIG. 7).

### Example 5

### Sensor Sensitivity

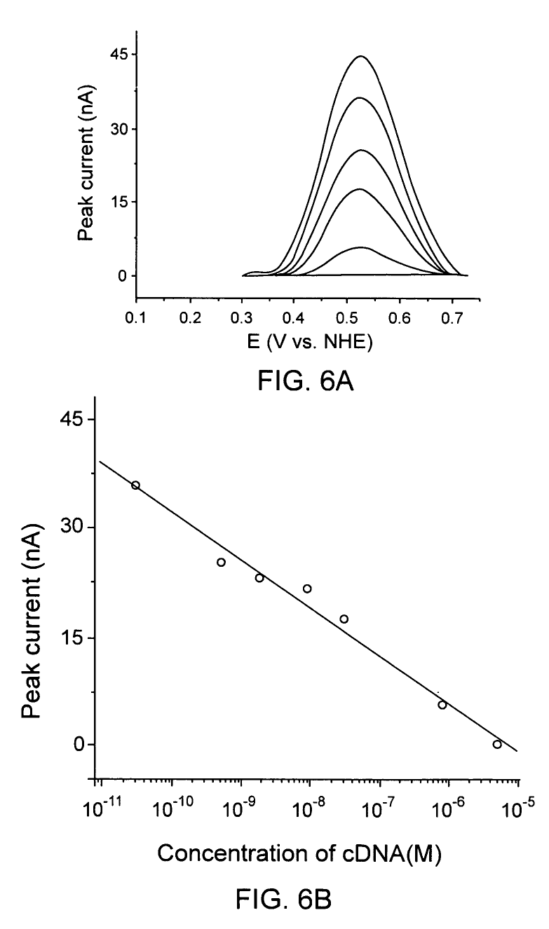

Employing a fixed 30-minute incubation time, the sensitivity of the sensor was tested. We observed readily measurable decreases in peak intensity at target DNA concentrations as low as 10 pM (FIG. 6). Peak currents were logarithmically related to target concentration across the almost six decade range of sample concentrations we investigated.

### Example 6

### Sensor Selectivity

The E-DNA sensor was highly selective. Employing a fixed 30-minute incubation time, we tested the sensitivity of the sensor. We observed readily measurable decreases in peak intensity at target DNA concentrations as low as 10 pM (FIG. 6a). Peak currents were logarithmically related to target concentration across the almost five decade range of sample concentrations we investigated (FIG. 6b).

No significant signal change was observed for electrodes incubated in DNA-free hybridization buffer or in the presence of the highest non-target DNA concentrations we investigated (10 M oligo 3, see Example 1). Thus the selectivity of the sensor relative to a random target sequence was in excess of 10⁶.

### Example 7

### Sensor Regeneration

The electrochemical DNA sensor was readily reusable. Washing the electrode with 1 M NaClO₄ at 95° C. and re-challenging with the target sequence, we successfully recovered up to about 80% of the original signal. The minor loss of the signal during recovery presumably resulted from the relative instability of ferrocene at high temperature. Other redox labels are more thermostable.

### Example 8

### Fabrication of the Stem-Loop DNA Structure with MB Label

Oligonucleotides were obtained from Synthegen (Houston, Tex.). The sensor oligonucleotide, 5′-HS—(CH2)6-GCGAGGT AAAACG ACGGCC AGTCTCGC-(CH2)6-NH2-3′ (oligo 1), contains a 5′ hexamethylene thiol and a 3′ hexamethylene amine. A methylene blue (MB) tag was conjugated to oligo 1 through coupling the succinimide ester of MB (MB-NHS, EMP Biotech, Germany) with the 5′ amine of oligo 1. The final product (oligo 1-MB) was purified by HPLC on a C18 column and confirmed by electrospray mass spectroscopy. The sequences of the target and control DNA oligos were 5′-ACTGGCCGTCGTTTTAC-3′ (oligo 2) and 5′-CGTATCATTGGACTGGC-3′ (oligo 3), respectively. Oligo 2 is fully complementary to the loop sequence while the control oligo 3 is a sequence unrelated to the probe sequence, which was used as the masking DNA.

### Example 9

### Preparation of the Functionalized Gold Electrode

Polycrystalline gold disks (1.6 mm diameter) (BAS Inc., West Lafayette, Ind.) were used as working electrodes. The E-DNA sensor was constructed by assembling the MB-labeled DNA stem-loop at the gold electrode. In order to construct the sensor as demonstrated in FIG. 4, a 0.1 mM solution of the stem-loop oligo 1-MB (with 100 mM NaCl, 5 mM MgCl₂ and 10 mM phosphate buffer at pH 7.0) was self-assembled on an extensively cleaned gold surface (Leopold, M. C., Black, J. A. and Bowden, E. F., Langmuir 18, 978-980 (2002); Fan, C., Gillespie, B., Wang, G., Heeger, A. J. and Plaxco, K. W., J. Phys. Chem. (B) 106, 11375-11383 (2002).). The prepared surface was subsequently passivated with excess 6-mercaptohexanol at 1 mM for about 2 hrs. The modified electrode was thoroughly rinsed, dried and then incubated in 1 M NaCl prior to use.

### Example 10

### Description of the MB Labeled E-DNA Sensor

Cyclic voltammetry (CV) and AC voltammetry (ACV) were performed at room temperature using a CHI 603 workstation (CH Instruments, Austin, Tex.). In ACV, we employ 10 Hz frequency and 25 mV amplitude. Potentials are reported versus the Ag/AgCl, 3 M NaCl reference electrode (BAS Inc.). A platinum wire was used as the counter electrode.

MB, as well as the previously employed ferrocene, is readily redoxable at gold electrodes. As demonstrated in FIG. 8, a pair of well-defined peaks were obtained for E-DNA in the absence of targets, which corresponds to the redox conversion of the MB label in close proximity to the gold electrode. Upon hybridization with complementary sequence to the loop range, the unfolding of the stem-loop moves the MB away from the electrode surface, which significantly decreases the electrochemical signal.

FIG. 8 provides a cyclic voltammogram for a gold electrode modified with the MB tagged, stem-loop oligonucleotide in the absence of target DNA (scan rate of 0.1 V/s). The electrolyte is 10 mM phosphate buffer/1 M NaCl, pH 7.0.

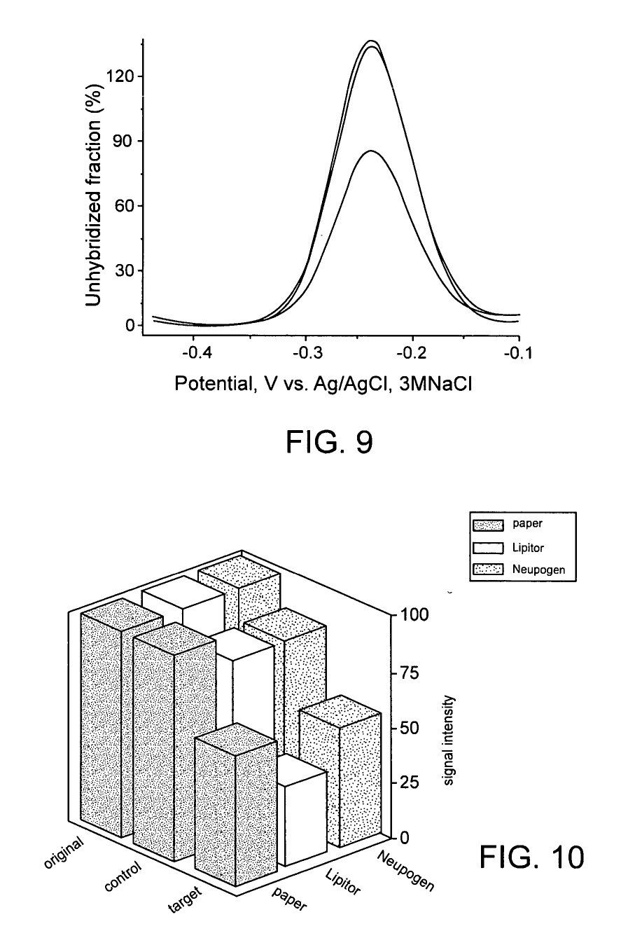

The MB-labeled E-DNA sensor works in alternating current voltammetry mode (ACV). ACV typically involves the application of a sinusodially oscillating voltage to an electrochemical cell which has proven to effectively reduce charging (background) current (O'Connor, S. D., Olsen, G. T. and Creager, S. E. J. Electroanal. Chem. 466, 197-202 (1999).). As shown in FIG. 9, the ACV of E-DNA has a nearly flat background, making the comparison between curves both convenient and quantitative. Consequently, ACV was used in the following DNA authentication studies.

FIG. 9 provides AC voltammograms for the E-DNA sensor before the test, and after the test with DNA microdots containing masking DNA (50 mg) only, and masking DNA (50 mg) mixed with target DNA (5 ng). The hybridization time was 30 minutes.

Use of the MB label results in at least three advantages. First, in the ferrocene labeled E-DNA sensor, the electrochemical experiments are best performed only in certain salt solutions (e.g., perchlorate), because ferrocene, if oxidized, is vulnerable to strong nucleophiles (e.g., chlorides) (Han, S. W., Seo, H., Chung, Y. K. & Kim, K., Langmuir 16, 9493-9500 (2000)). This limitation has been overcome via the employment of MB label, which is more stable in chloride solutions. Therefore, the use of MB labels not only avoids the risk of using potentially dangerous perchlorates, but avoids the necessity of removing possible chloride contaminations.

Second, ferrocene has little affinity for DNA strands, therefore the labeled ferrocene dangles under the stem-loop which may increase the surface heterogeneity. This effect is reflected by the non-ideal electrochemistry of ferrocene, such as decreased electron transfer rates and broadened peaks, due to dispersion of kinetic and thermodynamic parameters (rate constants, formal potentials etc.) (Saccucci, T. M. & Rusling, J. F. J. Phys. Chem. (B) 105, 6142-6147 (2001); Clark, R. A. & Bowden, E. F. Langmuir 13, 559-565 (1997)). In contrast, MB, a DNA intercalator, inserts into the stem double helix (Muller, W. and Crothers, D. M., Eur. J. Biochem. 54, 267-277 (1975); Boon, E. M., Salas, J. E. & Barton, J. K., Nat. Biotechnol. 20, 282-286 (2002)). Intercalation limits diffusion of the label, which leads to much improved electrochemical behavior, including sharper peaks (less thermodynamic dispersion) and smaller peak separations (less kinetic dispersion) (FIG. 1). For example, for CVs at 100 mV/s, the n×FWHM (full width at half-maximum) has been reduced from ˜170 mV to ˜140 mV, and the n×DE has been reduced from ˜60 mV to ˜30 mV in the MB labeled E-DNA (n stands for the electron transfer numbers).

Third, MB is very stable against thermal degradation in water and provides a more readily reusable sensor. This means that an MB-based sensor can be washed with hot water to remove hybridized target and give a good strong signal when reused.

### Example 11

### Document Authentication with E-DNA Sensor

The feasibility of encapsulating DNA sequence information in a piece of filter paper was tested. The E-DNA sensor was used as a convenient readout device. 1 ml of the DNA solution (˜5 ng oligo 2 with 10,000-fold excess of non-cognate DNA oligo 3) was added to a small circle (˜3 mm diameter) printed on filter paper with a ball pen. Interestingly, the DNA solution was confined in this cycle, possibly due to the fact that the diffusion of the solution in the filter paper was hindered by the hydrophobic pen ink. This DNA microdot, after being dried, was cut from the paper and immersed in 20 ml salt water containing 10 mM phosphate buffer with pH 7.0 and 1 M NaCl for approximately 10 min. 2 ml of the eluted solution was placed at the E-DNA electrode surface. After 30-min hybridization, the ACV signal dropped by about 40%. As a control, the E-DNA signal remain and almost unchanged in the case of a DNA microdot with only 50 mg masking DNA (oligo 3) (FIG. 9 and FIG. 10).

FIG. 10 provides comparisons among the E-DNA signals before and after counterfeiting test in filter paper, Lipitor and Neupogen.

This experiment clearly demonstrates the need for a very small amount of DNA oligo (˜5 ng), having the target sequence, to “authenticate” the provenance of documents. This sequence information can be read through an E-DNA sensor with the appropriate probe DNA. The extremely high specificity has enabled one to mask the sequence information in 10,000-fold excess of non-cognate “masking” DNA. The use of such a large excess of “masking” DNA will significantly impede counterfeiting efforts based on sequencing or cloning of the DNA authentication tag. Although this preliminary experiment was performed with filter paper, previous studies have proven it possible to encapsulate DNA in other substrates such as typical letter paper, with stability over two years at room temperature. However, in this case, time-consuming gel electrophoresis methods were used to obtain the results (Cook, L. J. & Cox, J. P. L. Biotechnol. Lett. 25, 89-94 (2003)). Given the complexity of DNA sequence information (a 17-mer corresponds to ˜seventeen billion combinations), convenience of encapsulation, and readout of the described technology, this DNA authentication technology is promising for authentication of important documents.

### Example 12

### Thwarting Drug Counterfeiting with E-DNA Sensor

Lipitor tablets were selected as an example of orally ingested drugs and Neupogen as an example of injectable drugs. Lipitor is a cholesterol lowering drug (Pfizer), while Neupogen (Amgen) is a cancer-control drug that fights against Neutropenia, a disease characterized by a low white blood cell count.

The Lipitor tablets were ground into powder and a droplet (approximately 1 μl) of DNA (20 ng oligo 2 with 200 mg masking DNA) was added to the powder. After drying in the air, the powder was dispersed in 50 ml salt water followed by filtering to obtain the supernatant. For the Neupogen liquid, 1 ml Neupogen was mixed with 1 μl DNA (20 ng oligo 2 with 200 mg masking DNA), and then diluted into a 50 ml solution; 2 ml of this solution was pipetted on the gold electrode surfaces. The control experiments were performed using only the masking DNA, in the absence of target DNA tag. As demonstrated in FIG. 10, we observed a significant decrease in the ACV signal after 30-min hybridization. In both cases, significantly smaller decreases of the corresponding signals were observed in the control experiments. The decreases in the control experiments possibly arise from the non-specific adsorption of some components of the drugs. It will be appreciated that one might wish to control the reaction time and the concentration of target DNA in order to obtain optimized results in actual sample detection. Nevertheless, due to the significant differences in response between the target DNA-containing experiments and the control experiments, we here demonstrated that it is possible to use the E-DNA sensor to read out the DNA information hidden in drugs.

### Example 13

### Reducing Background Signals with Additive Addition

The experiments set forth in Example 12 were repeated with one change. Glycerol (5% by volume) was present in the solutions pipetted onto the gold electrodes. The addition of glycerol greatly reduced the background signal in the control samples and resulted in the change in signal shown in FIG. 11. This experiment illustrates that the addition of materials which block nonspecific interactions between masking DNA and the probe produce a clearer and more specific result.

1. A detector for determining the presence of anoligonucleotide target having a target nucleotide sequence comprising;
an electrode capable of sensing redox events in a redox moiety and
an oligonucleotide probe immobilized on the electrode,
with at least one of the target and the probe comprising a redox moiety, the probe having a probe nucleotide sequence which hybridizes with the target nucleotide sequence and,
in the absence of hybridization between the target and the probe, at least one redox moiety being located in a first position relative to the electrode and, in the presence of hybridization between the target and the probe, said at least one redox moiety being located in a second position relative to the electrode, said first and second positions giving rise to distinguishable redox events detectable by the electrode wherein the second position is closer to the electrode than the first position.

2-3. (canceled)

4. The detector of claim 1 where one of the probe and the target comprises a redox moiety.

5. The detector of claim 1 wherein the probe comprises a redox moiety.

6. The detector of claim 1 wherein the target comprises a redox moiety.

7. The detector of claim 5 wherein the probe is immobilized on the electrode at a position distant from the redox moiety.

8. The detector of claim 1 wherein the electrode is capable of inducing redox events in the redox moiety.

9-11. (canceled)

12. The detector of claim 1 wherein the second configuration comprises internal hybridization between two regions in the probe.

13. The detector of claim 1 wherein the second configuration comprises a loop comprising a region of the target and a region of the probe.

14. The detector of claim 1 wherein the electrode comprises a metal.

15. The detector of claim 14 wherein the metal is gold.

16. The detector of claim 1 wherein the redox moiety is selected from the group consisting of purely organic redox labels, viologen, anthraquinone, ethidium bromide, daunomycin, methylene blue, and their derivatives, organo-metallic redox labels, ferrocene, ruthenium, bis-pyridine, tris-pyridine, bis-imidizole, and their derivatives, and biological redox labels, cytochrome c, plastocyanin, and cytochrome c′.

17-24. (canceled)

25. A detector for determining the presence of a oligonucleotide target having a target nucleotide sequence said detector comprising;
an electrode capable of sensing redox events in a redox moiety and
an oligonucleotide probe comprising a first region, a second region and a third region,
the first region being immobilized upon or proximate to the electrode,
the third region being bound to a redox moiety,
the second region being present in the probe intermediate the first and third regions and comprising a first nucleotide sequence which is complementary to and spaced apart from a second nucleotide sequence with which it self hybridizes to form a first loop which positions the redox moiety a first distance from the electrode, said first nucleotide sequence also hybridizing with the target nucleotide sequence in the target, such hybridizing with the target disrupting the first loop and permitting complementary nucleotide sequences in the second region to self hybridize to form a second loop which positions the redox moiety a second distance from the electrode, said first and second distances giving rise to distinguishable redox events detectable by the electrode wherein the second distance is shorter than the first distance.

26-27. (canceled)

28. The detector of claim 25 additionally comprising a detector for detecting electron transduction between the electrode and the redox moiety when the second loop is formed.

29. The detector of claim 28 additionally comprising an indicator for inducing electron transduction between the electrode and the redox moiety when the second loop is formed.

30. The detector of claim 29 wherein the first region is at one end of the probe.

31. The detector of claim 29 wherein the third region is at the second end of the probe.

32. The detector of claim 25 wherein the electrode comprises a metal.

33. The detector of claim 32 wherein the metal is gold.

34. The detector of claim 33 wherein the redox moiety is selected from the group consisting of purely organic redox labels, such as viologen, anthraquinone, ethidium bromide, daunomycin, methylene blue, and their derivatives, organo-metallic redox labels, such as ferrocene, ruthenium, bis-pyridine, tris-pyridine, bis-imidizole, and their derivatives, and biological redox labels, such as cytochrome c, plastocyanin, and cytochrome.

35. A detector for determining the presence of an oligonucleotide target having a target nucleotide sequence comprising;
an electrode capable of sensing redox events in a redox moiety and
an oligonucleotide probe immobilized on the electrode,
the target comprising a redox moiety, the probe having a probe nucleotide sequence which hybridizes with the target nucleotide sequence and,
in the absence of hybridization between the target and the probe, the redox moiety being located in a first position relative to the electrode and, in the presence of hybridization between the target and probe the redox moiety being located in a second position relative to the electrode, said first and second positions giving rise to distinguishable redox events detectable by the electrode.

36. The detector of claim 35 wherein the first position is closer to the electrode than the second position.

37. The detector of claim 35 wherein the second position is closer to the electrode than the first position.

38. The detector of claims 35 wherein the electrode is capable of inducing redox events in the redox moiety.

39. A method for detecting the presence of an oligonucleotide target having a target nucleotide sequence in a sample comprising:
obtaining a detector for determining the presence of an oligonucleotide target having a target nucleotide sequence comprising;
an electrode capable of sensing redox events in a redox moiety and
an oligonucleotide probe immobilized on the electrode
with at least one of the target and the probe comprising a redox moiety, the probe having a probe nucleotide sequence which hybridizes with the target nucleotide sequence and,
in the absence of hybridization between the target and the probe, at least one redox moiety being located in a first position relative to the electrode and, in the presence of hybridization between the target and the probe, said at least one redox moiety being located in a second position relative to the electrode, said first and second positions giving rise to distinguishable redox events detectable by the electrode,
contacting the sample under oligonucleotide hybridization conditions with the detector and sensing redox events in the redox moiety with the electrode in the presence of the sample and in the absence of the sample and,
correlating similarity in redox events between the two sensings with the absence of the target and a change in redox events with the presence of the target, wherein the target is associated with an object and wherein the sensing of the presence of the target is correlated with the authenticity of the object.

40. A method for detecting the presence of an oligonucleotide target having a target nucleotide sequence in a sample comprising:
obtaining a detector for determining the presence of an oligonucleotide target having a target nucleotide sequence comprising
an electrode capable of sensing redox events in a redox moiety and
an oligonucleotide probe immobilized on the electrode,
with at least one of the target and the probe comprising a redox moiety, the probe having a probe nucleotide sequence which hybridizes with the target nucleotide sequence and,
in the absence of hybridization between the target and the probe, at least one redox moiety being located in a first position relative to the electrode and, in the presence of hybridization between the target and the probe, said at least one redox moiety being located in a second position relative to the electrode, said first and second positions giving rise to distinguishable redox events detectable by the electrode,
contacting the sample under oligonucleotide hybridization conditions with the detector and sensing redox events in the redox moiety with the electrode and,
correlating the sensed redox event with at least one sensed redox even sensed in the presence of and/or the absence of the target wherein the target is associated with an object and wherein the sensing of the presence of the target is correlated with the authenticity of the object.

41-46. (canceled)

47. A method for detecting the presence of an oligonucleotide target having a target nucleotide sequence in a sample comprising:
obtaining a detector for determining the presence of a oligonucleotide target having a target nucleotide sequence said detector comprising;
an electrode capable of sensing redox events in a redox moiety and
an oligonucleotide probe comprising a first region, a second region and a third region,
the first region being immobilized upon or proximate to the electrode.
the third region being bound to a redox moiety,
the second region being present in the probe intermediate the first and third regions and comprising a first nucleotide sequence which is complementary to and spaced apart from a second nucleotide sequence with which it self hybridizes to form a first loop which positions the redox moiety a first distance from the electrode, said first nucleotide sequence also hybridizing with the target nucleotide sequence in the target, such hybridizing with the target disrupting the first loop and permitting complementary nucleotide sequences in the second region to self hybridize to form a second loop which positions the redox moiety a second distance from the electrode, said first and second distances giving rise to distinguishable redox events detectable by the electrode,
contacting the sample under oligonucleotide hybridization conditions with the detector and sensing redox events in the redox moiety with the electrode in the presence of the sample and in the absence of the sample and,
correlating similarity in redox events between the two sensings with the absence of the target and a change in redox events with the presence of the target wherein the target is associated with an object and wherein the sensing of the presence of the target is correlated with the authenticity of the object.

48. A method for detecting the presence of an oligonucleotide target having a target nucleotide sequence in a sample comprising:
obtaining a detector for determining the presence of a oligonucleotide target having a target nucleotide sequence said detector comprising;
an electrode capable of sensing redox events in a redox moiety and an oligonucleotide probe comprising a first region, a second region and a third region,
the first region being immobilized upon or proximate to the electrode,
the third region being bound to a redox moiety,
the second region being present in the probe intermediate the first and third regions and comprising a first nucleotide sequence which is complementary to and spaced apart from a second nucleotide sequence with which it self hybridizes to form a first loop which positions the redox moiety a first distance from the electrode, said first nucleotide sequence also hybridizing with the target nucleotide sequence in the target, such hybridizing with the target disrupting the first loop, and permitting complementary nucleotide sequences in the second region to self hybridize to form a second loop which positions the redox moiety a second distance from the electrode, said first and second distances giving rise to distinguishable redox events detectable by the electrode,
contacting the sample under oligonucleotide hybridization conditions with the detector and sensing redox events in the redox moiety with the electrode and,
correlating the sensed redox event with at least one sensed redox event sensed in the presence of and/or the absence of the target wherein the target is associated with an object and wherein the sensing of the presence of the target is correlated with the authenticity of the object.

49-50. (canceled)

51. A method for detecting the presence of an oligonucleotide target having a target nucleotide sequence in a sample comprising:
contacting the sample under oligonucleotide hybridization conditions with the detector of claim 35 and sensing redox events in the redox moiety with the electrode in the presence of the sample and in the absence of the sample and,
correlating similarity in redox events between the two sensings with the absence of the target and a change in redox events with the presence of the target.

52. A method for detecting the presence of an oligonucleotide target having a target nucleotide sequence in a sample comprising:
contacting the sample under oligonucleotide hybridization conditions with the detector of claim 35 and sensing redox events in the redox moiety with the electrode and,
correlating the sensed redox event with at least one sensed redox event sensed in the presence of and/or the absence of the target.

53. The method of claim 51 wherein the target is associated with an object and wherein the sensing of the presence of the target is correlated with the authenticity of the object.

54. The method of claim 52 wherein the target is associated with an object and wherein the sensing of the presence of the target is correlated with the authenticity of the object.

55. A method for authenticating an object comprising:
obtaining a detector for determining the presence of an oligonucleotide target having a target nucleotide sequence comprising;
an electrode capable of sensing redox events in a redox moiety and
an oligonucleotide probe immobilized on the electrode,
with at least one of the target and the probe comprising a redox moiety the probe having a probe nucleotide sequence which hybridizes with the target nucleotide sequence and,
in the absence of hybridization between the target and the probe, at least one redox moiety being located in a first position relative to the electrode and, in the presence of hybridization between the target and the probe, said at least one redox moiety being located in a second position relative to the electrode, said first and second positions giving rise to distinguishable redox events detectable by the electrode,
associating the object with the target,
sensing the presence of the target associated with the object; and
correlating the sensing of the presence of that target oligonucleotide with the authenticity of the object.

56. The method of claim 55 wherein the sensing is carried out in the presence of masking oligonucleotides.

57-58. (canceled)

59. A method for authenticating an object comprising:
obtaining a detector for determining the presence of a oligonucleotide target having a target nucleotide sequence said detector comprising;
an electrode capable of sensing redox events in a redox moiety and
an oligonucleotide probe comprising a first region, a second region and a third region,
the first region being immobilized upon or proximate to the electrode,
the third region being bound to a redox moiety,
the second region being present in the probe intermediate the first and third regions and comprising a first nucleotide sequence which is complementary to and spaced apart from a second nucleotide sequence with which it self hybridizes to form a first loop which positions the redox moiety a first distance from the electrode, said first nucleotide sequence also hybridizing with the target nucleotide sequence in the target, such hybridizing with the target disrupting the first loop and permitting complementary nucleotide sequences in the second region to self hybridize to form a second loop which positions the redox moiety a second distance from the electrode, said first and second distances giving rise to distinguishable redox events detectable by the electrode,
associating the object with the target,
sensing the presence of the target associated with the object; and
correlating the sensing of the presence of that target oligonucleotide with the authenticity of the object.

60. The method of claim 59 wherein the sensing is carried out in the presence of masking oligonucleotides.

61. A method for authenticating an object comprising:
obtaining a detector of claim 35,
associating the object with the target,
sensing the presence of the target associated with the object; and
correlating the sensing of the presence of that target oligonucleotide with the authenticity of the object.

62. The method of claim 61 wherein the sensing is carried out in the presence of masking oligonucleotides.
